(19) United States
(12) Patent Application Publication
(10) Pub. No.: US 2008/0014250 A1
(43) Pub. Date: Jan. 17, 2008

(54) Bifunctionalized polysaccharides

(75) Inventors: Gerard Soula, Meyzieu (FR); Remi Soula, Lyon (FR); Olivier Soula, Meyzieu (FR)

Correspondence Address: OLIFF & BERRIDGE, PLC, P.O. BOX 320850, ALEXANDRIA, VA, 22320-4850, US

(73) Assignee: PROTEINS AND PEPTIDES MANAGEMENT, LYON (FR)

(21) Appl. No.: 11/783,402

(22) Filed: Apr. 9, 2007

Related U.S. Application Data
(60) Provisional application No. 60/790,532, filed on Apr. 10, 2006.

(30) Foreign Application Priority Data
Apr. 7, 2006 (FR) 06/03130

(51) Int. Cl. A61K 9/70; A61K 31/28; A61K 31/295; A61K 31/30; A61K 31/315; A61P 17/00; A61P 9/00; C08B 37/02; A61P 25/00; A61K 31/715; A61K 38/00; A61K 38/16; A61K 38/18

(52) U.S. Cl. 424/443; 514/2; 514/494; 514/499; 514/501; 514/502; 514/54; 514/8; 536/51

(57) ABSTRACT

The present invention relates to a dextran and/or dextran derivative bifunctionalized by at least one imidazolyl radical Im and at least one hydrophobic group Hy, the said radical and the said group being each identical and/or different and grafted or bonded to the dextran and/or dextran derivative via one or more connecting arms R, Ri or Rh and functional groups F, Fi or Fh and the pharmaceutical compositions comprising one of the said dextrans and at least one active principle.

The present invention relates to novel biodegradable polymers based on polysaccharides and more particularly on dextrans.

These polymers are of use in particular for the administration of active principle(s) (APs) to man or to animals with a therapeutic and/or prophylactic purpose. These polymers can also serve to potentiate and protect endogenous active principles.

These polymers have been designed to correspond to several applications:
the systemic release of AP, such as proteins, for example insulin or growth hormone,
the local release of AP, such as growth factors, for example Transforming Growth Factors or Bone Morphogenic Proteins, in vitro cell culturing,
the in vivo implantation of cells,
healing in which the APs are endogenous growth factors.

Despite research carried out in these fields, many problems remain with regard to each of these applications. First of all, as regards the systemic release of proteins, it would be desirable to keep the concentration of the AP constant over a long period of time. In point of fact, in the case of the product Lantus for insulin, for example, the concentration can vary over the course of the day, which can result in hyperglycaemias. In the case of the administration of growth factors, which are powerful local therapeutic agents, one of the major problems consists in keeping them at their site of administration in order to prevent their systemic circulation, as is summarized in the paper by Seeherman, Cytokines and Growth Factors Reviews, 2005, 16, 329-345. In the case of cell culturing, the supply of growth factors is recognized to be beneficial. However, due to the low stability of these molecules in solution, large amounts of these expensive agents have to be used.

There thus exists an unsatisfied need for pharmaceutical compositions which make it possible:
to extend the release time of systemic APs, such as insulin or hGH,
to retain the active principle at the site of administration, for example in the case where the AP is a growth factor,
to limit the amount of AP, generally growth factors, employed in cell culturing,
to promote the action of endogenous APs, in particular in the case of healing.

Despite numerous attempts to develop novel polymers for achieving these medical objectives, only PLAGAs have been approved to date in the drug delivery field. These polymers form, in aqueous medium, dense solids which comprise the AP. In this case, the AP can be released over several weeks, which is one of the desired objectives. One formulation example is Nutropin Depot, developed by Alkermes and Genentech for the prolonged release (2 weeks) of the human growth hormone, described in Patent WO 95/29664.

However, this approach suffers from numerous weaknesses, such as:
a “burst” effect, that is to say that a significant portion of the AP is released immediately after injection,
chemical decomposition of the PLAGA polymer, which forms lactic and glycolic acids within the solid, which acids catalyse the decomposition of the polymer and, in some cases, that of the AP,
the local increase in the acidity is a source of inflammation. These two phenomena are described in the paper by Anderson, Adv. Drug Del. Rev., 1997, 28, 5-24.

For these reasons, Neutropin Depot has recently been withdrawn from the market.

Atrix describes, in U.S. Pat. No. 5,990,194, the use of PLAGA for the release of a peptide, leuprolide, under the name of Atrigel, which is a formulation based on an organic solvent. In addition to the problems related to PLAGA mentioned above, this technology exhibits the following failings:
The injection of an organic solvent. In the case of Atrigel, this solvent is classified among CMR compounds.
This system is difficult to apply to proteins because of the denaturing effects of NMP.

Other formulations with the aim of the controlled release of pharmaceutical APs employ crossable polymers but, despite numerous research efforts, no biomaterial involved in pharmaceutical compositions makes it possible to simultaneously solve the requirement of injectability and that of the retention at the site of administration of the active principle in order to control its systemic or local release.

The present invention relates to novel poly-saccharides and more particularly dextrans bifunctionalized by at least one imidazolyl radical Im and at least one hydrophobic group Hy which make it possible to satisfy the applications targeted above to which no solution has been found to date. Among functionalized polysaccharides, pectins (galacturonans), the acids of which are modified by amines and in particular an amine carrying an imidazole ring, are known from Patent WO 99/09067. These monofunctionalized polymers are not amphiphilic.

Other polysaccharides monofunctionalized by hydrophobic groups are known to a person skilled in the art. The studies by Akiyoski et al. (J. Controlled Release 1998, 54, 313-320) describe pullulans modified by cholesterol for the controlled release of insulin.

Dellacherie et al. describe hyaluronan derivatives modified by C₁₂ or C₁₈ fatty alkyl chains in Patent FR 2 794 763. This group was also described, in Patent FR 2 781 677, alginate derivatives modified by fatty alkyl chains.

Among functionalized dextrans, the carboxy-methyldextrans from Biodex described in U.S. Pat. No. 6,646,120 are modified by benzylamine, which is a hydrophobic group. These polymers are not functionalized by an imidazolyl radical.

Dellacherie et al. have also described dextrans functionalized by a hydrophobe (Durand, A. et al., Biomacromolecules, 2006, 7, 958-964.)(Durand, Alain et al., Colloid Polym. Sci., 2006, 284, 536-545.) which are obtained by reaction of the hydroxyl functional groups of the dextran with epoxides (phenyl glycidyl ether, 1,2-epoxyoctane or 1,2-epoxydodecane). The polymers described are thus not bifunctionalized and do not have an imidazolyl radical.

Bauer et al. describe dextrans functionalized by C₁₀ to C₁₄ fatty acids in U.S. Pat. No. 5,750,678. These polymers are also monofunctionalized.

A recent review of functional polymers based on dextrans (Heinze, Thomas et al., Adv. Polym. Sci., 2006, 205, 199-291) does not report a dextran bifunctionalized by a hydrophobe and an imidazolyl radical.

Compositions which are insoluble in water by chemical modification of anionic polysaccharides by nucleophiles are also known from WO 92/20349. Histidine and some of its derivatives appear among the nucleophiles but these polymers are monofunctional.

Thus, no bifunctionalized polysaccharide and more particularly no bifunctionalized dextran according to the invention is known from the prior art.

The invention thus relates to a polysaccharide bifunctionalized by at least one imidazolyl radical Im and at least one hydrophobic group Hy, the said radical and the said group being each identical and/or different and grafted or bonded to the polysaccharide via one or more connecting arms R, Ri or Rh and functional groups F, Fi or Fh.

In one embodiment, the polysaccharide according to the invention is chosen from the group consisting of hyaluronans, alginates, chitosans, galacturonans, chondroitin sulphate, dextrans, carboxymethyldextrans and carboxymethylcelluloses.

In one embodiment, the polysaccharide according to the invention is chosen from the group consisting of hyaluronans, alginates, chitosans and carboxymethyl-dextrans.

In one embodiment, the polysaccharide according to the invention is chosen from the group consisting of dextrans and carboxymethyldextrans.

The invention thus relates to a dextran and/or dextran derivative bifunctionalized by at least one imidazolyl radical Im and at least one hydrophobic group Hy, the said radical and the said group being each identical and/or different and grafted or bonded to the dextran and/or dextran derivative via one or more connecting arms R, Ri or Rh and functional groups F, Fi or Fh,
R represents a connecting arm composed of a chemical bond or of a chain comprising between 1 and 18 carbon atoms, optionally branched and/or unsaturated comprising one or more heteroatoms, such as O, N and/or S,
   R will be denoted Ri when it carries an imidazolyl radical and Rh when it carries a hydrophobic group, Ri and Rh being identical or different,
   F represents a functional group chosen from the ester, thioester, amide, carbonate, carbamate, ether, thioether or amine functional groups,
   F will be denoted Fi when it carries an imidazolyl radical and Fh when it carries a hydrophobic group, Fi and Fh being identical or different,
Im represents an imidazolyl radical, optionally substituted on one of the carbons by a C₁ to C₄ alkyl (Alky) group, of formula 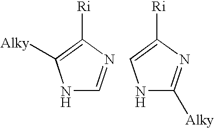
Hy represents a hydrophobic group chosen from the groups:
   linear or branched C₈ to C₃₀ alkyl, optionally unsaturated and/or comprising one or more heteroatoms, such as O, N or S,
   linear or branched C₈ to C₃₀ alkylaryl or arylalkyl, optionally unsaturated and/or optionally comprising a heteroatom,
   C₈ to C₃₀ polycyclic, optionally unsaturated,
the said dextran and/or dextran derivative being amphiphilic when it is in solution.

In one embodiment, it is amphiphilic at acidic pH.

In the continuation of the text, the term “dextran” is understood to mean, according to the invention, dextran and dextran derivatives.

The dextran derivatives are, in one embodiment, chosen from carboxylated derivatives.

The carboxylated derivatives of dextran are more particularly chosen from carboxymethyldextrans and the reaction products between succinic anhydride and dextran.

According to the invention, the bifunctionalized dextran and/or dextran derivative can correspond to the following general formulae:

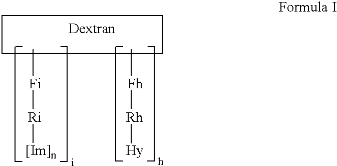

n is between 1 and 3,

i represents the molar fraction of imidazolyl radical with respect to one monosaccharide unit, of between 0.1 and 0.9,

h represents the molar fraction of hydrophobic group with respect to one monosaccharide unit, of between 0.01 and 0.5,

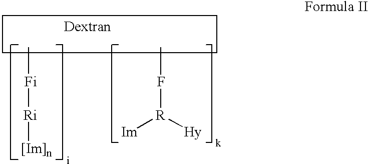

n is between 1 and 3,

i represents the molar fraction of imidazolyl radical with respect to one monosaccharide unit, of between 0 and 0.9,

k represents the molar fraction of hydrophobic group with respect to one monosaccharide unit, of between 0.01 and 0.5.

Surprisingly, the dextrans bifunctionalized by at least one imidazolyl radical and at least one hydrophobic group according to the invention solidify at physiological pH while making possible the retention of the AP in the polymer. Thus, the active principles are retained at the site of injection in vivo without being either decomposed or denatured.

The term “solidification” is understood to mean that the polymer can either form a solid or form a hydrogel.

A hydrogel is a type of colloid obtained in an aqueous medium in which a liquid comprises a solid forming a fine network which extends throughout the system. The solid and liquid phases are continuous therein.

Two types of bifunctionalized dextrans correspond to this invention, cationic dextrans and anionic dextrans.

The cationic dextrans according to the invention have the property of forming a homogeneous solution in a pH range of less than 6 and of solidifying at a pH close to physiological pH.

At a pH close to physiological pH, all or part of the charged and thus hydrophilic imidazole segments will be converted to neutral segments, which results in its solidification.

This effect of solidification can be combined with an effect of physical crosslinking by coordination of the imidazolyl rings of the polymer, and imidazolyl rings possibly present on the active principle, with polyvalent transition metals, such as zinc. This coordination takes place only at a pH of greater than 6.

The anionic dextrans according to the invention have the property of forming a homogeneous solution at neutral pH and of solidifying at a pH close to physiological pH in the presence of transition metal salts.

When they form a homogeneous solution, the dextrans according to the invention are amphiphilic and thus dissolved in the form of micelles and/or of nanoparticles.

At a pH close to physiological pH, the solidifying brought about by a physical crosslinking effect takes place by coordination of the imidazolyl rings of the polymer, and imidazolyl rings possibly present on the active principle, with polyvalent transition metals, such as zinc.

The following scheme represents the mode of action of the metal salts at physiological pH with the imidazoles carried by the polymer or the AP.

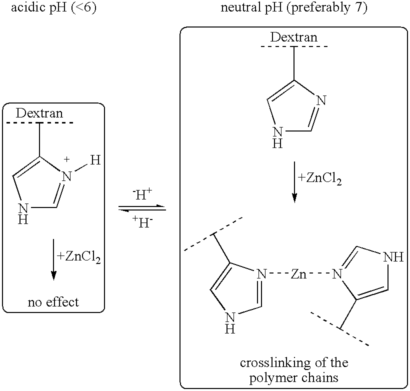

Depending on the properties of the polymers which make it possible to obtain solidifications at physiological pH, the injectable formulations will be prepared in the pH regions in which the said polymers form a homogeneous solution.

In one embodiment, the dextran and/or dextran derivative according to the invention is characterized in that the Ri group, when it is not a bond, is chosen from the following groups:

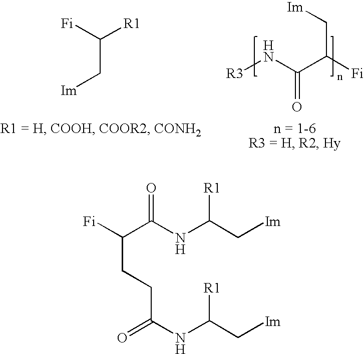

R2 being chosen from alkyl radicals comprising from 1 to 18 carbon atoms.

In one embodiment, the dextran and/or dextran derivative according to the invention is characterized in that the Ri group is a bond.

In one embodiment, the dextran and/or dextran derivative according to the invention is characterized in that the imidazole-Ri group is chosen from the groups obtained by grafting a histidine ester, histidinol, histidinamide or histamine.

These imidazole derivatives can be represented as follows:

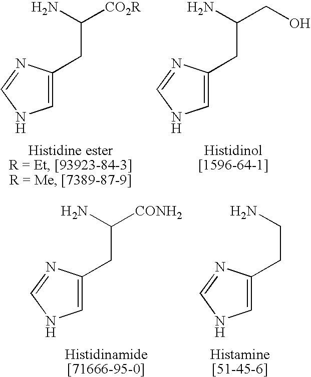

In one embodiment, the dextran and/dextran derivative according to the invention is characterized in that Hy will be chosen from the group consisting of fatty acids, fatty alcohols, fatty amines, cholesterol derivatives, including cholic acid, and phenols, including α-tocopherol.

In one embodiment, the dextran and/or dextran derivative according to the invention is characterized in that the Rh group, when it is not a bond, is chosen from the groups:

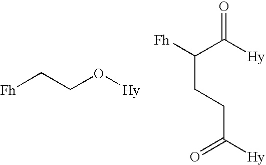

In one embodiment, the dextran and/or dextran derivative according to the invention is characterized in that the Rh group is a bond.

In one embodiment, the dextran and/or dextran derivative according to the invention is characterized in that the Ri group, when it is not a bond, is chosen from the groups:

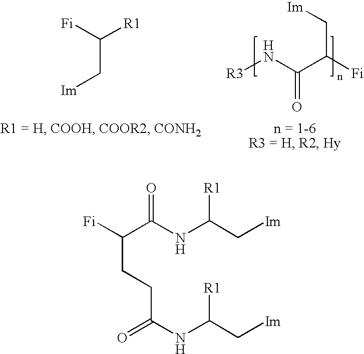

R2 being chosen from alkyl radicals comprising from 1 to 18 carbon atoms,

and the Rh group is a bond.

In one embodiment, the dextran and/or dextran derivative according to the invention is characterized in that the imidazole-Ri group is chosen from histidine esters, histidinol, histidinamide or histamine.

In one embodiment, the dextran and/or dextran derivative according to the invention is characterized in that Hy will be chosen from the group consisting of fatty acids, fatty alcohols, fatty amines, cholesterol derivatives, including cholic acid, phenols, including α-tocopherol, and hydrophobic amino acids.

The hydrophobic amino acids are chosen from tryptophan derivatives, such as tryptophan ethyl ester, phenylalanine derivatives, leucine derivatives, valine derivatives or isoleucine derivatives.

The dextran and/or dextran derivative can have a degree of polymerization m of between 10 and 10 000.

In one embodiment, it has a degree of polymerization m of between 10 and 1000.

In another embodiment, it has a degree of polymerization m of between 10 and 500.

The invention also relates to a pharmaceutical composition comprising one of the dextrans and/or dextran derivatives according to the invention as described above and at least one active principle.

The term “active principle” is understood to mean a product in the form of a single chemical entity or in the form of a combination having a physiological activity. The said active principle can be exogenous, that is to say that it is contributed by the composition according to the invention. It can also be endogenous, for example growth factors, which will be secreted in a wound during the first phase of healing and which may be retained on the said wound by the composition according to the invention.

The invention also relates to a pharmaceutical composition comprising one of the dextrans and/or dextran derivatives according to the invention as defined above and a transition metal salt.

In one embodiment, the transition metal is chosen from the group consisting of zinc, iron, copper and cobalt.

The invention also relates to a pharmaceutical composition according to the invention as defined above, characterized in that it is provided in the form of a homogeneous solution or of a suspension in water at a pH of less than 6.

The invention also relates to a pharmaceutical composition according to the invention as defined above, characterized in that the homogeneous solution and/or the suspension at a pH of less than 6 is composed of micelles and/or nanoparticles. The term “nanoparticles” is understood to mean objects in suspension in water, the mean diameter of which is less than 600 nm.

The invention also relates to a pharmaceutical composition according to the invention as defined above, characterized in that it is provided in the form of a suspension of microparticles in water at a pH close to physiological pH. The term “microparticles” is understood to mean objects, the mean diameter of which is greater than 600 nm, and the term “pH close to physiological pH” is understood to mean a pH of between 6 and 8.

The invention also relates to a pharmaceutical composition according to the invention as described above, characterized in that it can be administered intravenously, intramuscularly, intraosseously, subcutaneously, transdermally or ocularly.

The invention also relates to a pharmaceutical composition according to the invention as described above, characterized in that it can be administered orally, nasally, vaginally or buccally.

The invention also relates to a pharmaceutical composition according to the invention as described above, characterized in that is provided in a solid form.

The invention also relates to a pharmaceutical composition according to the invention as described above, characterized in that it is obtained by solidification controlled by the pH.

In one embodiment, the solidification is carried out at a pH of greater than 6.5.

The invention also relates to a pharmaceutical composition according to the invention as described above, characterized in that it is obtained by drying and/or lyophilization.

The invention also relates to a pharmaceutical composition according to the invention as described above, characterized in that it can be administered in the form of a stent, film or coating of implantable biomaterials, or implant.

The invention also relates to a composition as described above, characterized in that it undergoes physical crosslinking at the site of injection.

The invention also relates to a composition as described above, characterized in that it makes possible the retention of the active principle at the site of injection.

The pharmaceutical compositions according to the invention are obtained by conventional pharmaceutical formulating techniques known to a person skilled in the art and will be prepared either industrially or at the time of use.

The invention also relates to a pharmaceutical composition according to the invention as described above, characterized in that the active principle is chosen from the group consisting of proteins, glycoproteins, peptides and nonpeptide therapeutic molecules.

According to the invention, the proteins or glycoproteins are chosen from hormones, such as insulin or hGH, from growth factors, such as the members of the superfamily of the Transforming Growth Factors-β (TGF-β), such as Bone Morphogenic Proteins (BMP), Platelet Derived Growth Factors (PDGF), Insulin Growth Factors (IGF), Nerve Growth Factors (NGF), Vascular Endothelial Growth, Factors (VEGF), Fibroblasts Growth Factors (FGF), Epidermal Growth Factors (EGF), cytokines of the interleukin (IL) or interferon (IFN) type.

Mention may be made, among the therapeutic medical applications, of:
the local release of AP, such as growth factors, for example TGFs, BMPs, PDGFs, NGFs, VEGFs, IGFs, FGFs or EGFs,
the systemic release of AP, such as proteins, for example insulin, growth hormone, EPO, ILs or IFNs,
the in vivo administration of cells,
in vitro cell culturing,
healing in which the APs are endogenous growth factors.

The compositions according to the invention as described above in which the active principle is chosen from the group consisting of proteins, glycoproteins, peptides and nonpeptide therapeutic molecules comprise between 0.005% and 2% by weight of active principle, with respect to the total weight of the composition.

In one embodiment, the compositions comprise between 0.01% and 0.5% by weight of proteins, glycoproteins, peptides and nonpeptide therapeutic molecules, with respect to the total weight of the composition.

Targeted among the medical applications cited in the local release of growth factors, in particular BMPs, are local treatments of bones weakened by osteoporosis. These treatments consist in regenerating the bones quickest to be broken and subjected to high physical stresses, in particular the hips, wrists and vertebra. These treatments are both curative, in the case of fractures, and preventive, in high-risk situations.

The invention thus relates to the use of the dextrans and/or dextran derivatives and/or of the compositions according to the invention in the treatment or formulation of medicaments intended for local treatments of bones weakened by osteoporosis.

In this specific case, the composition comprises between 0.005% and 2% of BMP, with respect to the total weight of the composition.

In one embodiment, the composition comprises between 0.01% and 0.5% of BMP, with respect to the total weight of the composition.

Targeted among the medical applications cited in the local release of growth factors, in particular of NGFs or TGFs-β, are the treatments for the regeneration of nervous tissues.

The invention thus relates to the use of the dextrans and/or dextran derivatives and/or of the compositions according to the invention in the treatment or formulation of medicaments intended for the regeneration of nervous tissues.

In this specific case, the composition comprises between 0.005% and 2% of NGF or of TGF-β, with respect to the total weight of the composition.

In one embodiment, the composition comprises between 0.01% and 0.5% of NGF or of TGF-β, with respect to the total weight of the composition.

Targeted among the medical applications cited in the local release of growth factors, in particular of TGF-β, PDGFs or VEGFs, are the treatments for the regeneration of cardiovascular tissues.

The invention thus relates to the use of the dextrans and/or dextran derivatives and/or of the compositions according to the invention in the treatment or the formulation of medicaments intended for the regeneration of cardiovascular tissues.

In this specific case, the composition comprises between 0.005% and 2% of VEGF or TGF-β, with respect to the total weight of the composition.

In one embodiment, the composition comprises between 0.01% and 0.5% of VEGF or TGF-β, with respect to the total weight of the composition.

Targeted among the medical applications cited in the local release of growth factors, in particular of PDGFs or FGFs, are the treatments for the regeneration of skin tissues.

The invention thus relates to the use of the dextrans and/or dextran derivatives and/or of the compositions according to the invention in the treatment or formulation of medicaments intended for the regeneration of skin tissues.

In this specific case, the composition comprises between 0.005% and 2% of PDGF or FGF, with respect to the total weight of the composition.

In one embodiment, the composition comprises between 0.01% and 0.5% of PDGF or FGF, with respect to the total weight of the composition.

According to the invention, the proteins are chosen from the group consisting of insulin or growth hormone hGH.

According to the invention, the nonpeptide therapeutic molecules are chosen from the group consisting of anticancers, such as taxol or cisplatin.

In this specific case, the composition comprises between 0.005% and 2% of insulin or growth hormone hGH, with respect to the total weight of the composition.

In one embodiment, the composition comprises between 0.01% and 0.5% of insulin or growth hormone hGH, with respect to the total weight of the composition.

According to the invention, the active principle is chosen from the group of the peptides chosen from leuprolide or short sequences of ParaThyroid Hormone (PTH).

The pharmaceutical compositions according to the invention are provided either in the liquid form (nanoparticles or microparticles in suspension in water or in mixtures of solvents) or in the powder, implant, film, gel or cream form.

In the case of local and systemic releases, the modes of administration envisaged are subcutaneously, intradermally, intramuscularly, orally, nasally, vaginally, ocularly, buccally, and the like.

The pharmaceutical compositions according to the invention can thus be employed to form an implant comprising one or more pharmaceutical active principles for their controlled release over a long period of time. This application is particularly advantageous in the treatment of solid tumours with an anticancer or in cell regeneration.

The invention also relates to a pharmaceutical composition physically crosslinked at the site of injection comprising an active principle chosen from the group consisting of proteins, glycoproteins, peptides and nonpeptide therapeutic molecules.

The invention also relates to the use of the bifunctionalized dextrans and/or dextran derivatives according to the invention in the preparation of pharmaceutical compositions, such as described above.

### EXAMPLE 1

### Synthesis of a Carboxymethyldextran Modified by Histidine Ethyl Ester and Benzylamine

The acid functional groups of a carboxymethyldextran (mean acid degree per glycoside unit of 1.0) are activated in the presence of N-MethylMorpholine and of isobutyl chloroformate in NMP. Benzylamine and then histidine ethyl ester are grafted to this activated polymer. The polymer obtained has the following structure: 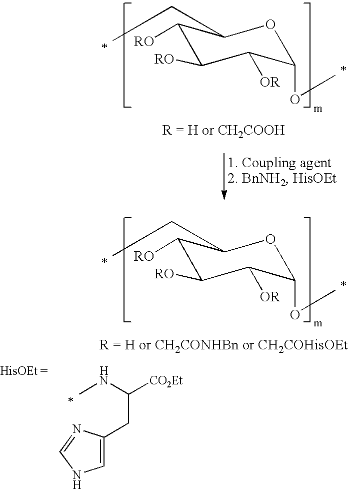
The level of acid functional groups modified by:

histidine ethyl ester is 55%,

benzylamine is 45%.

The level of unmodified acids is zero.

### EXAMPLE 2

### Synthesis of a Carboxymethyldextran Modified by Histidine Ethyl Ester and Dodecylamine

The active functional groups of a carboxymethyldextran (mean acid degree per glycoside unit of 1.0) are activated in the presence of N-MethylMorpholine and of isobutyl chloroformate in DMF. Histidine ethyl ester and dodecylamine are grafted to this activated polymer. The polymer obtained has the following structure: 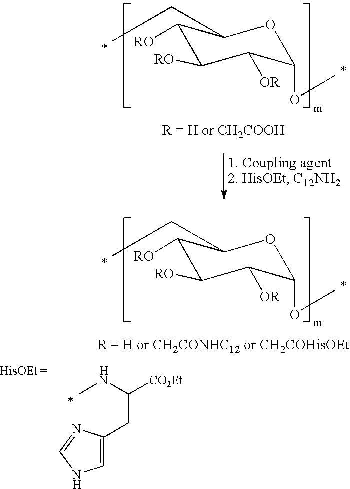
The level of acid functional groups modified by:

histidine ethyl ester is 85%,

dodecylamine is 10%.

The level of unmodified acids is 5%.

### EXAMPLE 3

### Synthesis of a Carboxymethyldextran Modified by Histidinamide and Benzylamine

The acid functional groups of a carboxymethyldextran (mean acid degree per glycoside unit of 1.0) are activated in the presence of N-MethylMorpholine and of isobutyl chloroformate in NMP. Histidinamide and benzylamine are grafted to this activated polymer. The polymer obtained has the following structure: 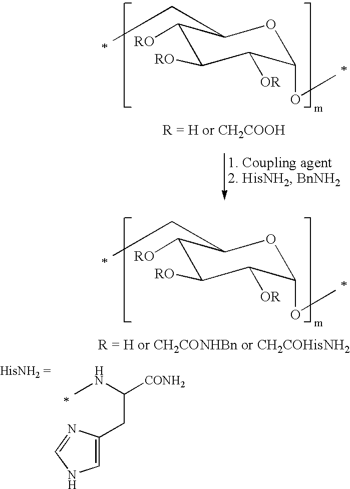
The level of acid functional groups modified by:

histidinamide is 65%,

benzylamine is 30%.

The level of unmodified acids is 5%.

### EXAMPLE 4

### Synthesis of a Carboxymethyldextran Modified by Histidine Ethyl Ester and Tryptophan Ethyl Ester

The acid functional groups of a carboxymethyldextran (mean acid degree per glycoside unit of 1.0) are activated in the presence of N-MethylMorpholine and of isobutyl chloroformate in DMF at 0° C. Histidine ethyl ester and tryptophan ethyl ester are grafted to this activated polymer. The polymer obtained is characterized by a level of acid functional groups modified by:

histidine ethyl ester of 70%,

tryptophan ethyl ester of 30%.

The level of unmodified acids is zero.

### EXAMPLE 5

### Synthesis of a Carboxymethyldextran Modified by Histidine Ethyl Ester and Tryptophan Ethyl Ester

The acid functional groups of a carboxymethyldextran (mean acid degree per glycoside unit of 0.7) are activated in the presence of N-MethylMorpholine and of isobutyl chloroformate in DMF at 0° C. Histidine ethyl ester and tryptophan ethyl ester are grafted to this activated polymer. The polymer obtained is characterized by a level of acid functional groups modified by:

histidine ethyl ester of 60%,

tryptophan ethyl ester of 40%.

The level of unmodified acids is zero.

### EXAMPLE 6

### Synthesis of a Carboxymethyldextran Modified by Histidine Ethyl Ester and Benzylamine

The acid functional groups of a carboxymethyldextran (mean acid degree per glycoside unit of 1.0) are activated in the presence of N-MethylMorpholine and of isobutyl chloroformate in DMF at 0° C. Histidine ethyl ester and benzylamine are grafted to this activated polymer. The polymer obtained has the following structure: 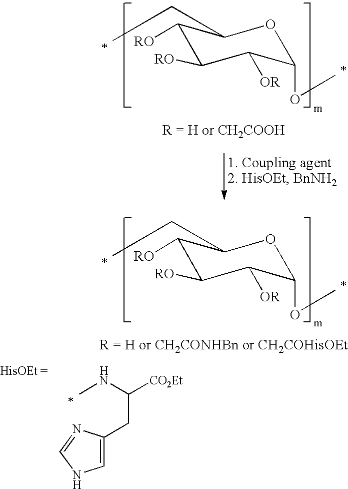
The level of acid functional groups modified by:

histidine ethyl ester is 10%,

benzylamine is 45%.

The level of unmodified acids is 45%.

### EXAMPLE 7

### Synthesis of a Carboxymethyldextran Modified by Histidine Ethyl Ester and Benzylamine

The acid functional groups of a carboxymethyldextran (mean acid degree per glycoside unit of 1.0) are activated in the presence of N-MethylMorpholine and of isobutyl chloroformate in DMF at 0° C. Histidine ethyl ester (0.2 equivalent with respect to the acids) and benzylamine (0.45 equivalent with respect to the acids) are grafted to this activated polymer. The polymer obtained has the following structure: 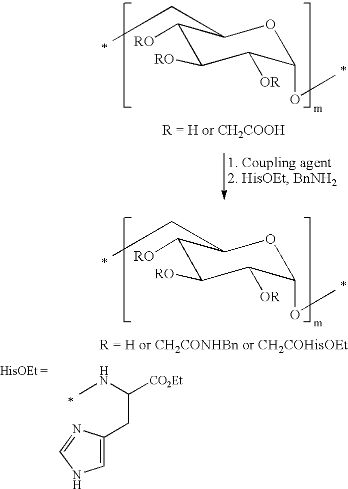
The level of acid functional groups modified by:

histidine ethyl ester is 30%,

benzylamine is 45%.

The level of unmodified acids is 25%.

### EXAMPLE 8

### Synthesis of a Carboxymethyldextran Modified by Benzylamine

The polymer is prepared according to U.S. Pat. No. 6,646,120. The level of acid functional groups modified by benzylamine is 40%.

### EXAMPLE 9

### Study of the Solubility of the Polymers as a Function of the pH

The polymers described in the preceding Examples (1 to 8) were dissolved at acidic pH, pH of less than 6. The state of the solutions of polymers at acidic pH is described in the second column in the table. Subsequently, these solutions at acidic pH are dispersed in a medium buffered to neutral pH (PBS buffer). The state of the solutions at neutral pH is described in the third column in the table.

| | Bifunctionalized | At acidic pH | At neutral pH |
|---|---|---|---|
| Polymer | dextran | (<6) | (pH = 7.2) |
| 1-5 | Cationic | Homogeneous | Two-phase |
| | | and fluid | medium |
| 6-7 | Anionic | | Homogeneous |
| | | | and fluid |
| 8 | | Homogeneous | Homogeneous |
| | | and fluid | and fluid |

In the case of the polymers obtained in Examples 1 to 5, two phases coexist at neutral pH, one of solidified polymer and a clear aqueous solution. In the case of the polymers obtained in Examples 6 and 7, there is only a single phase, which is a homogeneous and more dilute solution of polymer. The absence of or the low functionalization of the polymer by the imidazole rings does not make possible solidification at neutral pH.

### EXAMPLE 10

### Study of the Solidification in the Presence of ZnCl₂

The polymers described in the preceding Examples (1 to 8) were dissolved in water, at acidic pH for the polymers 1 to 5 and at neutral pH for the polymers 6 to 8. For the polymers 1 to 5, ZnCl₂ is added to the polymer solution at acidic pH. The ZnCl₂ number is 1 per 2 imidazoles. The solutions at acidic pH of the polymers 1 to 5 comprising ZnCl₂ are dispersed in a medium buffered to neutral pH (PBS buffer). The solutions of the polymers 6 to 8 at neutral pH are dispersed in a medium buffered to neutral pH (PBS buffer) comprising ZnCl₂. The state of the solutions of polymers at neutral pH in the presence of ZnCl₂ is described in the third column in the table.

| Polymer | At acidic pH (<6) + ZnCl₂ | At neutral pH |
|---|---|---|
| 1-5 | Homogeneous and fluid | Two-phase medium |
| 6-7 | | Two-phase medium |
| 8 | | Homogeneous and fluid |

The physical crosslinking by coordination of the zinc by the imidazoles of the polymers which are obtained in Examples 1 to 5 results in an as effective, indeed even enhanced, solidification. The polymers 6 and 7 precipitate in the presence of the zinc ions at neutral pH, whereas they do not precipitate in the absence of these ions. The polymer obtained in Example 8, which was soluble whatever the pH, is insensitive to the presence of transition metal salt. The low functionalization of the polymer by the imidazole rings makes possible solidification via the ZnCl₂ at neutral pH. On the other hand, the absence of the functionalization of the polymer by the imidazole rings does not make possible solidification via the ZnCl₂ at neutral pH.

### EXAMPLE 11

### Study of the Distribution of the Transition Metal Salts between the Solid and the Solution

The distribution of the zinc(II) chloride was studied in order to be able to demonstrate that all the metal salts were indeed trapped in the polymer phase which has solidified at physiological pH. The solution of this salt is colourless. The polymer obtained in Example 1, dissolved at acidic pH, is treated with half an equivalent of ZnCl₂ with respect to the imidazoles. The solution is homogeneous and colourless. This solution is then dispersed in a medium buffered to neutral pH (PBS buffer). The precipitate which instantaneously forms is white and the supernatant is clear and colourless. The latter is analysed by solids content and confirms the absence of zinc salt in this phase. This demonstrates the quantitative trapping of the metal salts in the solid formed by the polymer at neutral pH. In the case of the polymer obtained in Example 8, the homogeneous solution obtained at neutral pH comprises zinc salts.

### EXAMPLE 12

### Study of the Sequestration of a Protein in the Solid Formed by the Polymer at Neutral pH

The sequestration of cytochrome C, a red protein, in the polymer phases at physiological pH was studied. For this, a solution of the polymer obtained in Example 1 at acidic pH was prepared (30 mg/ml). Cytochrome C was dissolved at 10 mg/ml. The solution of this protein is red. 2 mg of the protein are added to 30 mg of the polymer obtained in Example 1 dissolved at acidic pH. The solution is homogeneous and red. This solution is then dispersed in a medium buffered to neutral pH (PBS buffer). The precipitate which instantaneously forms is red, whereas the supernatant is clear and colourless. This demonstrates the quantitative sequestration of the protein in the solid formed by the polymer at neutral pH.

This experiment was successfully repeated for other polymers. On the other hand, in the case of the polymer obtained in Example 8, the solution at neutral pH is red and does not comprise precipitate.

### EXAMPLE 13

### Study of the Sequestration of a Protein in the Solid Formed by the Polymer at Neutral pH in the Presence of ZnCl₂

The sequestration of cytochrome C in solids at physiological pH was studied in the presence of ZnCl₂. For this, a solution of the polymer obtained in Example 1 at acidic pH was prepared (30 mg/ml). Cytochrome C was dissolved at 10 mg/ml. The solution of this protein is red. The ZnCl₂ was dissolved at 13.6 mg/ml. 2 mg of the protein and 6.8 mg of ZnCl₂ are added to 30 mg of the polymer obtained in Example 1 dissolved at acidic pH. The acidic solution is homogeneous and red. This solution is then dispersed in a medium buffered to neutral pH (PBS buffer). The precipitate which instantaneously forms is red, whereas the supernatant is clear and colourless. This demonstrates the quantitative sequestration of the protein in the solid formed by the polymer at neutral pH in the presence of ZnCl₂.

This experiment was successfully repeated for the polymers obtained in Examples 1-7. The metal salt thus makes it possible to form a solid which traps the protein by virtue of the physical crosslinking of the polymer chains by zinc/imidazole coordination.

### EXAMPLE 14

### Study of the Sequestration of PDGF-BB in the Solid Formed by the Polymer at Neutral pH in the Presence of ZnCl₂

The sequestration of PDGF-BB in the solid at physiological pH was studied in the presence of ZnCl₂. For this, a solution of the polymer obtained in Example 1 was prepared at acidic pH (20 mg/ml). 0.02 mg of PDGF-BB and 6.5 mg of ZnCl₂ are added to 100 μl of the solution of polymer at acidic pH. The acidic solution is homogeneous and clear. This solution is then dispersed in a medium buffered to neutral pH (10 volumes of 30 mM PBS buffer). The precipitate rapidly forms. The PDGF-BB present in the supernatant is quantitatively determined by ELISA after centrifuging the heterogeneous medium. The concentration of PDGF-BB in the supernatant is less than 0.2 μg/ml, whereas it is 2 μg/ml in the polymer-free control. There is therefore indeed virtually quantitative sequestration of the protein, of greater than 90%, in the solid formed by the polymer at neutral pH in the presence of ZnCl₂.

### EXAMPLE 15

### Polymer Obtained in Example 1+BMP-2 Formulation

A solution No. 1 of the polymer obtained in Example 1 at a concentration of 50 mg/m¹ is prepared at pH 5. A solution No. 2 of BMP-2 at a concentration of 1 mg/ml is prepared at pH 7. The osmolarity of each solution is adjusted to 300 mOsm by the addition of NaCl. These solutions are stored at 4° C. One hour before the injection into the neck of the femur of a patient, a solution No. 3 is prepared by mixing 0.9 ml of the solution No. 1 and 0.1 ml of the solution No. 2. The solution obtained is homogeneous and has a pH close to 5.

### EXAMPLE 16

### Polymer Obtained in Example 1+ZnCl₂+BMP-2 Formulation

The solutions Nos. 1 and 2 as described in Example 15 have their osmolarity adjusted by addition of ZnCl₂. The final solution No. 3 is then prepared at the time of use in the way described in Example 15.

### EXAMPLE 17

### Polymer Obtained in Example 1+BMP-2 Formulation

The final solution No. 3 as described in Example 15 can be prepared at the time of use from the lyophilized polymer obtained in Example 1 and from lyophilized BMP-2. The osmolarity of the final solution is adjusted to 300 mOsm by addition of NaCl. The polymer has a buffering power and results in a solution having a pH close to 5. This final solution is clear.

### EXAMPLE 18

### Polymer Obtained in Example 1+ZnCl₂+BMP-2 Formulation

The final solution No. 3 as described in Example 15 can be prepared at the time of use from the lyophilized polymer obtained in Example 1 and from lyophilized BMP-2. In this case, the osmolarity of the final solution is adjusted to 300 mOsm by addition of ZnCl₂. This final solution is clear.

### EXAMPLE 19

### Polymer Obtained in Example 1+BMP-2 Formulation

The formulation can be prepared at the time of use by the dissolution of 0.1 mg of lyophilized BMP-2 in 1 ml of solution of polymer obtained in Example 1 at 45 mg/ml, at pH 5 and adjusted to 300 mOsm by the addition of NaCl. This final solution is clear.

### EXAMPLE 20

### Polymer Obtained in Example 1+ZnCl₂+BMP-2 Formulation

The solution of polymer obtained in Example 1 at 45 mg/ml and at pH 5 as described in Example 17 is adjusted to 300 mOsm by the addition of ZnCl₂. 0.1 mg of lyophilized BMP-2 is dissolved in 1 ml of polymer solution at the time of use. This final solution is also clear.

### EXAMPLE 21

### Polymer Obtained in Example 1+PDGF-BB Formulation

This case concerns the preparation of a formulation formed of polymer obtained in Example 1 and of PDGF-BB. This formulation is prepared according to one of the six methods described in Examples 15 to 20. The formulation comprises 45 mg of polymer and 0.1 mg of PDGF-BB per 1 ml of solution. This solution is clear and has a pH close to 5.

This formulation is employed in the treatment of foot ulcers of diabetic patients.

### EXAMPLE 22

### Polymer Obtained in Example 1+hGH Formulation

This case concerns the preparation of a formulation formed of polymer obtained in Example 1 and of hGH. This formulation is prepared according to one of the six methods described in Examples 15 to 20. The formulation comprises 45 mg of polymer and 5 mg of hGH per 1 ml of solution. This solution is clear and has a pH close to 5.

This formulation is injected in patients once weekly.

1. Dextran and/or dextran derivative bifunctionalized by at least one imidazolyl radical Im and at least one hydrophobic group Hy, the said radical and the said group being each identical and/or different and grafted or bonded to the dextran and/or dextran derivative via one or more connecting arms R, Ri or Rh and functional groups F, Fi or Fh, characterized in that:
R represents a connecting arm composed of a chemical bond or of a chain comprising between 1 and 18 carbon atoms, optionally branched and/or unsaturated comprising one or more heteroatoms, such as O, N and/or S,
R will be denoted Ri when it carries an imidazolyl radical and Rh when it carries a hydrophobic group, Ri and Rh being identical or different,
F represents a functional group chosen from the ester, thioester, amide, carbonate, carbamate, ether, thioether or amine functional groups,
F will be denoted Fi when it carries an imidazolyl radical and Fh when it carries a hydrophobic group, Fi and Fh being identical or different,
Im represents an imidazolyl radical, optionally substituted on one of the carbons by a C₁ to C₄ alkyl (Alky) group, of formula 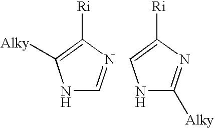
Hy represents a hydrophobic group chosen from the groups:
linear or branched C₈ to C₃₀ alkyl, optionally unsaturated and/or comprising one or more heteroatoms, such as O, N or S,
linear or branched C₈ to C₃₀ alkylaryl or arylalkyl, optionally unsaturated and/or optionally comprising a heteroatom,
C₈ to C₃₀ polycyclic, optionally unsaturated,
the said dextran and/or dextran derivative being amphiphilic when it is in solution.

2. Dextran and/or dextran derivative according to claim 1, characterized in that the dextran derivatives are chosen from carboxylated derivatives.

3. Dextran and/or dextran derivative according to claim 2, characterized in that the carboxylated dextran derivatives are chosen from carboxymethyldextrans and the reaction products between succinic anhydride and dextran.

4. Dextran and/or dextran derivative according to claim 1, characterized in that it corresponds to the general formula I: 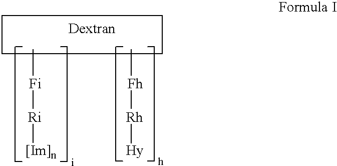
n is between 1 and 3,
i represents the molar fraction of imidazolyl radical with respect to one monosaccharide unit, of between 0.1 and 0.9,
h represents the molar fraction of hydrophobic group with respect to one monosaccharide unit, of between 0.01 and 0.5.

5. Dextran and/or dextran derivative according to claim 1, characterized in that it corresponds to the general formula II: 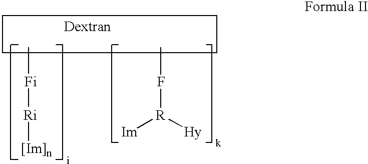
n is between 1 and 3,
i represents the molar fraction of imidazolyl radical with respect to one monosaccharide unit, of between 0 and 0.9,
k represents the molar fraction of hydrophobic group with respect to one monosaccharide unit, of between 0.01 and 0.5.

6. Dextran and/or dextran derivative according to claim 1, characterized in that the Ri group, when it is not a bond, is chosen from the groups: 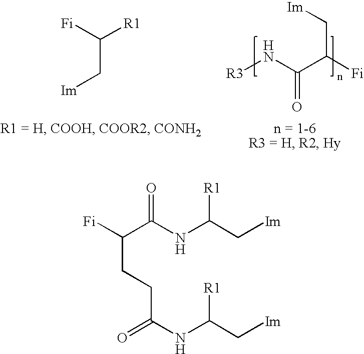
R2 being chosen from alkyl radicals comprising from 1 to 18 carbon atoms.

7. Dextran and/or dextran derivative according to claim 1, characterized in that the Ri group is a bond.

8. Dextran and/or dextran derivative according to claim 1, characterized in that the Rh group, when it is not a bond, is chosen from the groups:

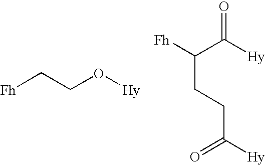

9. Dextran and/or dextran derivative according to claim 1, characterized in that the Rh group is a bond.

10. Dextran and/or dextran derivative according to claim 1, characterized in that the Ri group is chosen from the groups: 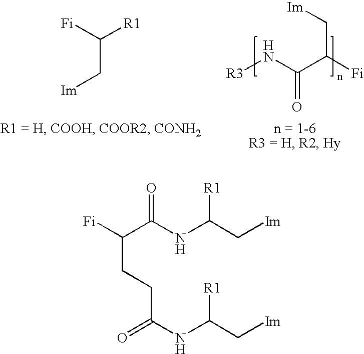
R2 being chosen from alkyl radicals comprising from 1 to 18 carbon atoms, and the Rh group is a bond.

11. Dextran and/or dextran derivative according to claim 1, characterized in that the imidazole-Ri group is chosen from histidine esters, histidinol, histidinamide or histamine.

12. Dextran and/or dextran derivative according to claim 1, characterized in that Hy is chosen from the group consisting of fatty acids, fatty alcohols, fatty amines, cholesterol derivatives, including cholic acid, phenols, including α-tocopherol, and hydrophobic amino acids.

13. Pharmaceutical composition comprising one of the polysaccharides according to claim 1 and at least one active principle.

14. Pharmaceutical composition comprising any one of the polysaccharides according to claim 1 and a transition metal salt.

15. Pharmaceutical composition according to claim 14, characterized in that the transition metal is chosen from the group consisting of zinc, iron, copper and cobalt.

16. Pharmaceutical composition according to claim 13, characterized in that it is provided in the form of a homogeneous solution or of a suspension in water at a pH of less than 6.5.

17. Pharmaceutical composition according to claim 16, characterized in that the homogeneous solution and/or the suspension is composed of micelles and/or of nanoparticles.

18. Pharmaceutical composition according to claim 13, characterized in that it is provided in the form of a suspension of microparticles in water at a pH close to physiological pH.

19. Pharmaceutical composition according to claim 13, characterized in that it can be administered intravenously, intramuscularly, intraosseously, subcutaneously, transdermally or ocularly.

20. Pharmaceutical composition according to claim 13, characterized in that it can be administered orally, nasally, vaginally or buccally.

21. Pharmaceutical composition according to claim 13, characterized in that it is provided in the solid form.

22. Pharmaceutical composition according to claim 21, characterized in that it is obtained by solidification controlled by the pH at a pH of greater than 6.

23. Pharmaceutical composition according to claim 21, characterized in that it undergoes a physical crosslinking at the site of injection.

24. Pharmaceutical composition according to claim 21, characterized in that it makes possible the retention of the active principle at the site of injection.

25. Pharmaceutical composition according to claim 21, characterized in that it is obtained by drying and/or lyophilization.

26. Pharmaceutical composition according to claim 21, characterized in that it can be administered in the form of a stent, film or coating of implantable biomaterials, implant, gel or cream.

27. Pharmaceutical composition according to claim 13, characterized in that the active principle is chosen from the group consisting of proteins, glycoproteins, peptides and nonpeptide therapeutic molecules.

28. Pharmaceutical composition according to claim 25, characterized in that the proteins or glycoproteins are chosen from growth factors, such as the members of the superfamily of the Transforming Growth Factors-β (TFG-β), such as Bone Morphogenic Proteins (BMP), Platelet Derived Growth Factors (PDGF), Insulin Growth Factors (IGF), Nerve Growth Factors (NGF), Vascular Endothelial Growth Factors (VEGF), Fibroblasts Growth Factors (FGF), Epidermal Growth Factors (EGF), cytokines of the type of the Interleukins (IL) or Interferons (INF).

29. Pharmaceutical composition according to claim 27, characterized in that the active principle is chosen from the group of peptides chosen from leuprolide or short sequences of ParaThyroid Hormone PTH.

30. Pharmaceutical composition according to claim 27, characterized in that the active principle is chosen from the group of the nonpeptide therapeutic molecules, such as anticancers, for example taxol or cisplatin.

31. Pharmaceutical composition according to claim 27, characterized in that the active principle is chosen from the group consisting of insulin or growth hormone hGH.

32. Treatment method or method for the formulation of medicaments intended for the regeneration of nervous tissues, characterized in that it comprises the use of dextrans and/or dextran derivatives according to claim 1.

33. Treatment method or method for the formulation of medicaments intended for the regeneration of cardiovascular tissues, characterized in that it comprises the use of dextrans and/or dextran derivatives according to claim 11.

34. Use of the dextrans and/or dextran derivatives and/or of the compositions according to claim 1 in the treatment or the formulation of medicaments intended for the regeneration of skin tissues.
